(19) United States
(12) Patent Application Publication
(10) Pub. No.: US 2022/0008599 A1
(43) Pub. Date: Jan. 13, 2022

(54) LIQUID DIFFUSER AND LIQUID DIFFUSING SYSTEM

(71) Applicant: MISCATO LIMITED, Hong Kong (CN)

(72) Inventors: Michelle ., Hong Kong (CN); Mingyu KIM, Hong Kong (CN); Myungsun PARK, Hong Kong (CN)

(21) Appl. No.: 17/294,696

(22) PCT Filed: Dec. 5, 2019
(86) PCT No.: PCT/CN2019/123188

§ 371 Date: May 18, 2021

(30) Foreign Application Priority Data
Dec. 5, 2018 (IB) PCT/IB2018/059648

(51) Int. Cl. A61L 9/14

(52) U.S. Cl. CPC A61L 9/14; A61L 2209/111; A61L 2209/14; A61L 2209/134; A61L 2209/12

(57) ABSTRACT

In one aspect, provided is a liquid diffuser comprising a base, a cover and at least one duct. According to another aspect, provided is a liquid diffuser system comprising an electronic device and at least one liquid diffuser. Other example embodiments are also described. In certain embodiments, the liquid diffusers allow easy, single-hand manageable, leakage-proof and hygienic replacement of the liquid containers. In certain embodiments, the liquid diffusers provides a smart system to manage or control the time for diffusion and the type of liquid to be used in response to user's requirement and/or data collected from the environment.

### CROSS-REFERENCE TO RELATED APPLICATION

This application claims priority to, and the benefit of, PCT international application PCT/IB2018/059648 filed 5 Dec. 2018, entitled Liquid Diffusing System. The entire contents of the foregoing application are hereby incorporated by reference for all purposes.

### FIELD OF INVENTION

This invention relates to a liquid diffuser and a liquid diffusing system thereof.

### BACKGROUND OF INVENTION

Diffusion of aromatic liquid in the form of aromatherapy can be useful for a number of reasons such as relieving stress, aiding sleep, increasing energy, or affecting mood. Traditional liquid dispensing systems for aromatherapy often use additional materials or media to aid in the dispensation of the liquids such as oils or essences. Even systems that don't use additional materials or media still have various issues, such as clogging, sanitation, or ease of use. There are many devices on the market but there is a need for further improvement.

### SUMMARY OF INVENTION

In the light of the foregoing background, it is an object of the present invention to provide an improved liquid diffuser and liquid diffusing system thereof.

An example embodiment of the present invention relates to the field of spraying mist, and more particularly, to a system for dispensing mist bearing substances including scent, nutraceutical, pharmaceutical, flavoring, medicine, and the wireless control of the dispensation in the air.

In one aspect, the object of the present invention is to provide a liquid diffusion system for users, which not only can provide clean and fresh mists via nebulizing mechanism, but also can be remotely controlled by a handset or other devices. Moreover, this system can control various diffusers at one time.

To achieve the above object, in certain example embodiments, the following technical solutions are adopted: a liquid diffusing system comprising a diffuser for diffusing liquid which comprises: a base having a cavity for accommodating a liquid container and a platform for connecting the container and applying external force; a container located in the cavity of the base for containing liquid; a chamber, one end of which is connected to the container and the other end of which is connected to the platform; a nozzle connected with the chamber for sucking up liquid from the container by a tube; a sealing member engaged with the chamber for sealing the chamber and the nozzle; and wherein the system further comprises an air source connected to the platform for flowing air into the chamber. After air flows into the nozzle, the nozzle makes low pressure by Bernoulli's principle, and the liquid flows into the nozzle and turned into micro-particles by the low pressure when external force is applied on sealing between the nozzle and the chamber, and the micro-particles are sprayed outside of the system.

In another example embodiment, preferably, the system further comprises a locking member for locking the sealing state between the chamber and the nozzle.

In another example embodiment, in addition, the system further comprises a trigger for triggering the locking member to change the state between the chamber and the nozzle from releasing to sealing, or change the state between the chamber and the nozzle from sealing to releasing.

In another example embodiment, preferably, the air source is an actuator providing air current into the container. And preferably, the actuator is an air pump.

In another example embodiment, according to the present invention, the system further comprises a tray located in the cavity of the base for receiving a plurality of containers. Preferably, the tray is provided with at least one protruding member at the bottom, and the base is provided with at least one slot for receiving the protruding members.

In another example embodiment, according to the liquid diffusing system of the present invention, the platform includes at least one hole for spraying micro-particles generated by the system.

In another example embodiment, according to the liquid diffusing system of the present invention, the system further comprises a wireless system for controlling the liquid diffusing system, in which the wireless system comprises: a diffuser hardware comprising controller, a memory, a communication module and an actuator; a remote control interface for controlling comprising a communication module, a controller and a controlling App, which is configured to communicate with the diffuser hardware so as to be preprogrammed to activate said diffuser according to a predetermined activation profile and deactivated when wireless connection between said communication is interrupted.

In another example embodiment, preferably, the wireless system is configured to control one diffuser at a time or multiple diffusers at once based on a predetermined schedule, selection or profile as commanded by user through user interface.

In another example embodiment, preferably, the wireless system is configured to control one or more than one liquid container to be diffused based on predetermined schedule, selection or profile as commanded by user through user interface.

In another example embodiment, preferably the diffuser hardware is operated by parameters which comprises frequency of liquid dispensing action to control intensity.

In another example embodiment, more preferably, the wireless system further comprises cloud as an intermediary between the diffuser and the remote control interface. More preferably, the wireless system further comprises a light unit for providing light. More preferably, the wireless system further comprises a music unit for providing music. More preferably, the wireless system further comprises an air filter for filtering air.

According to one embodiment of the invention, the wireless system further comprises a sensor for collecting data so as to monitor the status of the diffuser or the environment. Preferably, the sensor includes air quality sensor, air flow sensor, humidity and load sensor. The data points of the liquid diffusing system comprise of data from sensors, timer, user input to the user interface, and user behavior when using the system.

According to one embodiment of the invention is where the data collected through the diffuser hardware and remote control interface is feed into an algorithm, intelligent algorithm, machine learning system, or other forms of artificial intelligent systems to generate output data that is presented back to admin or user interface. The output is also stored in the cloud.

According to another embodiment of the invention, the remote control interface is communicated with a timer configured to activate and deactivate the diffuser according to user instruction. The system of the invention does not use additional material to aid in the spraying of liquid, which only adopts Bernoulli principle, a theorem of fluid dynamics that states that as the speed of air current increases, simultaneously the pressure drops. By the principle, the liquid in the container can be turned into micro-particles due to sudden decrease in pressure. Thus only providing air in the diffuser results in the operation of the diffuser.

In another example embodiment, the system can include a plurality of containers to be operated at the same time, which can generate diversity of mist in one diffuser.

In another example embodiment, the system can be operated by user through a user interface via wireless connection, which enables remote and automated control of the system and that has not been possible with diffusers in the prior art.

In another example embodiment, additional accessories such as light, music speaker, air filter and sensors are provided in the system so that additional complementary features can be obtained, if desired.

These and other objectives of the present invention will no doubt become obvious to those of ordinary skill in the art after reading the following detailed description of the preferred embodiment, which is illustrated in the various figures and drawings.

In another aspect, provided is a liquid diffuser which includes a base, a cover and at least one duct. The base has a cavity therein to accommodate at least one liquid container with at least one nozzle. The at least one duct extended through the cover. Each duct being connectable in gas communication to each nozzle. Each duct further includes a gas stream line connectable from a gas source to the corresponding nozzle such that a gas stream is in direct contact with a liquid inside the liquid container to create a mist via the nozzle.

In one example embodiment, the cover is engageable with the base via one or more locking members configured to bias the cover between an open state in which the one of more liquid containers are accessible and a sealed state in which the base and the cover are in gas communication.

In one example embodiment, each gas stream line is connected in gas communication to a separate actuator.

In one example embodiment, the liquid diffuser further includes a single actuator connectable to the gas stream lines. In one further example embodiment, the liquid diffuser further includes a trigger to selectively control and direct the gas stream from the actuator to at least one designated nozzle. In one further example embodiment, comprising a controller to control the actuator for regulating the gas stream, and/or the trigger for selecting the designated nozzle.

In one example embodiment, each duct further includes a sealing member to seal at least a portion of the nozzle.

In one example embodiment, the base further comprises a slidably removable tray to receive the at least one liquid container.

In one example embodiment, the cover further includes at least one vent. Each duct connects in gas communication with a corresponding vent such that the mist is diffused through the corresponding vent.

In another aspect, provided is a liquid diffuser which includes a base having a cavity therein to accommodate at least one liquid container with at least one nozzle; a cover that includes one or more ducts that corresponds to the nozzles; an actuator for generating a gas stream; a trigger for controlling and directing the gas stream to at least one designated nozzle; a wireless transceiver for receiving the command signals from an electronic device via a network; and a controller for generating control signals based on command signals received from an electronic device. The control signals are provided to the actuator for regulating the gas stream, and/or to the trigger for selecting the designated nozzle, so that intensity of liquid diffusion and choice of the liquid to be diffused can be controlled remotely.

In one example embodiment, the liquid diffuser further includes one or more sensors for sensing one or more parameters of the environment and/or status of the liquid diffuser; wherein the controller generates the command signals based on the parameters.

In one example embodiment, the sensors include one or more of an air quality sensor, an air flow sensor, a humidity sensor, a temperature sensor and a load sensor.

In another aspect, provided is a liquid diffusing system which includes an electronic device for sending command signals via a network; and at least one liquid diffuser. Each liquid diffuser has an unique identity and includes: one or more liquid containers, each container having one nozzle; a cover that includes one or more vents that corresponds to the nozzles; an actuator for generating a gas stream; a trigger for controlling and directing the gas stream to at least one designated nozzle; a wireless transceiver for receiving the command signals from the electronic device via the network; and a controller for generating control signals based on the command signals. The control signals are provided to the actuator for regulating the gas stream, and/or to the trigger for selecting the designated nozzle, so that intensity of liquid diffusion and choice of the liquid to be diffused can be controlled remotely.

In one example embodiment, the liquid diffuser further includes one or more sensors for sensing one or more parameters of the environment and/or status of the liquid diffuser; wherein the controller generates the command signals based on the parameters.

In one example embodiment, the sensors include one or more of an air quality sensor, an air flow sensor, a humidity sensor, a temperature sensor and a load sensor.

In one example embodiment, the liquid diffusing system further includes a server, wherein the server communicates with the electronic device and the liquid diffuser. The server receives data from the electronic device and/or the liquid diffuser, analyzing the data and provides feedback to the electronic device and/or the liquid diffuser.

In one example embodiment, the electronic device further includes a processor, a memory that stores one or more groups of pre-determined command signals for the liquid diffuser; and a wireless transmission module. The processor determines one or more groups of the pre-determined command signals based on a time schedule and the wireless transmission module sends the one or more groups of pre-determined command signals to at least one liquid diffuser.

In one example embodiment, the liquid diffusing system further includes one or more additional devices that communicate with the electronic device. The additional device is a light unit, a music unit and/or an air filter. The one or more additional devices are activated in response to the command signals received from the electronic device.

There are many advantages to the present invention. In certain embodiments, the liquid diffusers have a lower tendency to clog at the nozzles and therefore provide clean mists and minimize the maintenance. In certain embodiments, the liquid diffusers allow easy, single-hand manageable, leakage-proof and hygienic replacement of the liquid containers. In certain embodiments, the liquid diffusers provides a smart system to manage or control the time for diffusion and the type of liquid to be used in response to a user's requirement and/or data collected from the environment. For example, inhalation of Lavandula angustifolia vapor is suitable for evenings as it lowers anxiety and promotes restful sleep. However, it is not suitable for mornings or when users want to concentrate.

### BRIEF DESCRIPTION OF FIGURES

For a more complete understanding of the present invention, reference is made to the following detailed description of various example embodiments thereof, considered in conjunction with the accompanying drawings, in which:

FIG. 1 is an exploded schematic view of liquid diffusing system; of the present invention;

FIG. 2 is a schematic view of the present invention showing the connection of the chamber, the nozzle and the container;

FIG. 3 is a schematic view of the tray and the base of the present invention;

FIG. 4 is a schematic view of the present invention showing the actuator and the tray;

FIG. 5 is a block diagram describing wireless control of a diffuser via a remote control interface;

FIG. 6 is a block diagram describing wireless control of a plurality of diffusers via a remote control interface;

FIG. 7 is a block diagram showing details of the diffuser hardware and the remote control interface;

FIG. 8 is a block diagram showing details of part of the diffuser hardware and the remote control interface;

FIG. 9 is a block diagram showing details the diffuser hardware, a cloud and the remote control interface;

FIG. 10 is an app screenshot showing one embodiment of details of user interface that user can use to remotely control the system;

FIG. 11 is an app screenshot showing one embodiment of details of user interface where user can set a predetermined profile of how to schedule the system;

FIG. 12 is an app screenshot showing one embodiment of details of user interface where user can review the stored profiles and modify the predetermined profile; and

FIG. 13 is an app screenshot showing one embodiment of details of user interface where user inputs information, which would become one of the inputs of the data analysis/machine learning algorithm.

FIG. 14 is a schematic perspective view of a liquid diffuser in accordance with one example embodiment.

FIG. 15 is a perspective-exploded view of a liquid diffuser in accordance with one example embodiment.

FIG. 16 is a perspective-exploded view of a container, a nozzle, a duct assembly and a gas supply line of a liquid diffuser, in accordance with one example embodiment.

FIG. 17 is a cross-sectional side view of a nozzle and a duct in accordance with one example embodiment.

FIG. 18A is a cross-sectional horizontal view of a liquid diffuser in accordance with one example embodiment.

FIG. 18B is another cross-sectional horizontal view of the liquid diffuser of FIG. 18A in accordance with the same example embodiment.

FIG. 19A-19D are a series of different cross-sectional side views of the liquid diffuser showing how the diffuser works and the gas flows, in accordance with an example embodiment.

FIG. 20 shows a liquid diffusing system 2000 according to an example embodiment.

FIG. 21 shows a liquid diffusing system 2100 according to an example embodiment.

FIG. 22 shows a liquid diffusing system 2200 according to an example embodiment.

### DETAILED DESCRIPTION

As used herein and in the claims, “comprising” means including the following elements but not excluding others.

As used herein and in the claims, “liquid diffuser” refers to a device to disperse liquids, such as essential oils, into the surroundings or atmosphere. In certain embodiments, such device is electrically controllable.

As used herein and in the claims, “couple” refers to electrical coupling or connection either directly or indirectly via one or more electrical means unless otherwise stated.

As used herein and in the claims, “connect” refers to physical binding or connecting with other elements either directly or indirectly.

As used herein and in the claims, “base” refers to a structure having a cavity to accommodate the containers and is intended in a broad generic sense, and does not indicate any particular shape or type of structure.

As used herein and in the claims, “platform” or “cover” refers to a structure placing over and optionally engageable with a base and is intended in a broad generic sense, and does not indicate any particular shape or type of structure.

As used herein and in the claims, “chamber” or “duct” refers to a passage or a channel by which a gas or other substance is conveyed and is intended in a broad generic sense, and does not indicate any particular shape or type of structure.

As used herein and in the claims, “nozzle” refers to a structure to disperse a liquid into small droplets. In certain embodiments, a nozzle may have an inlet for gas stream line and at least one outlet for generating a mist.

As used herein and in the claims, “actuator” refers to something that directly or indirectly actuates an internal or external structure (such as a pump) to supply a gas source.

As used herein and in the claims, “trigger” refers to something that can directly or indirectly control and direct a gas stream line to one or more selected channels.

As used herein and in the claims, “mist” refers to small droplets of one or more liquids able to suspend in a gas stream.

As used herein and in the claims, “schedule” refers to instructions or parameters to control a certain parts or device.

In the above description, certain terms may be used such as “top,” “bottom,” “transverse,” “vertical,” and the like. These terms are used, where applicable, to provide ease of description to explain with relative relationships. However, these terms are not intended to imply absolute relationships, positions, and/or orientations. For example, with respect to an object, a “top” surface can become a “bottom” surface simply by turning the object over.

As used herein, the phrase “at least one”, when used with a list of items, means different combinations of one or more of the structures may be used and only one of the structures in the list may be necessary. The structure may be a particular object, thing, or category.

### Example 1

The present invention discloses a liquid diffuser system comprising one or more liquid containers to be dispensed. The liquid diffusion is performed by turning liquid into micro-particles with air pressure based on Bernoulli theorem. This system could be also remotely controlled from a user interface through wireless connection, creating a predetermined schedule, selection or profile, and able to control one or multiple systems. The wireless control further has the ability to control the device's diffusing mechanism including selecting one or more liquid containers at one time, diffusing selected liquid from liquid containers, and tuning the intensity of diffusion by changing the frequency of diffusion. The invention further has data collection and analysis capabilities, the output of which is presented back to admin or user interface.

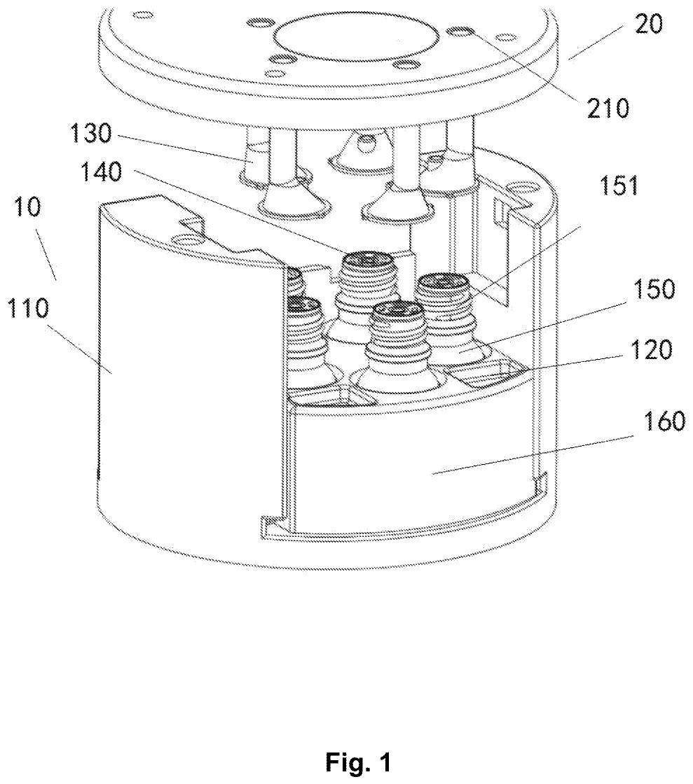

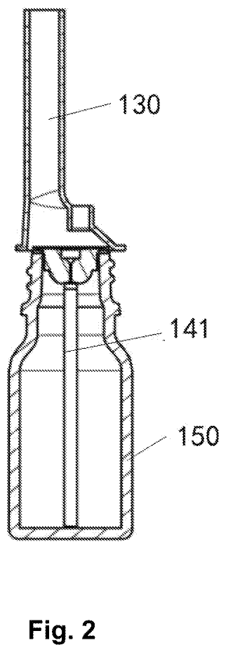

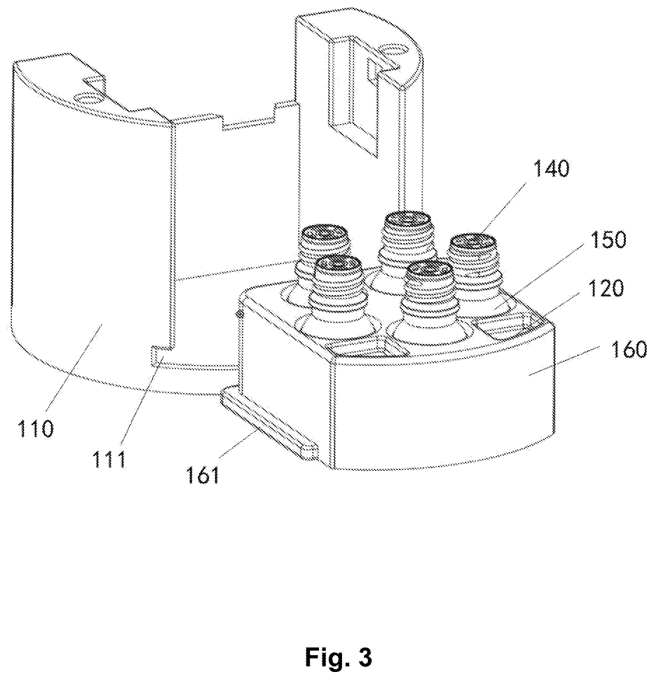

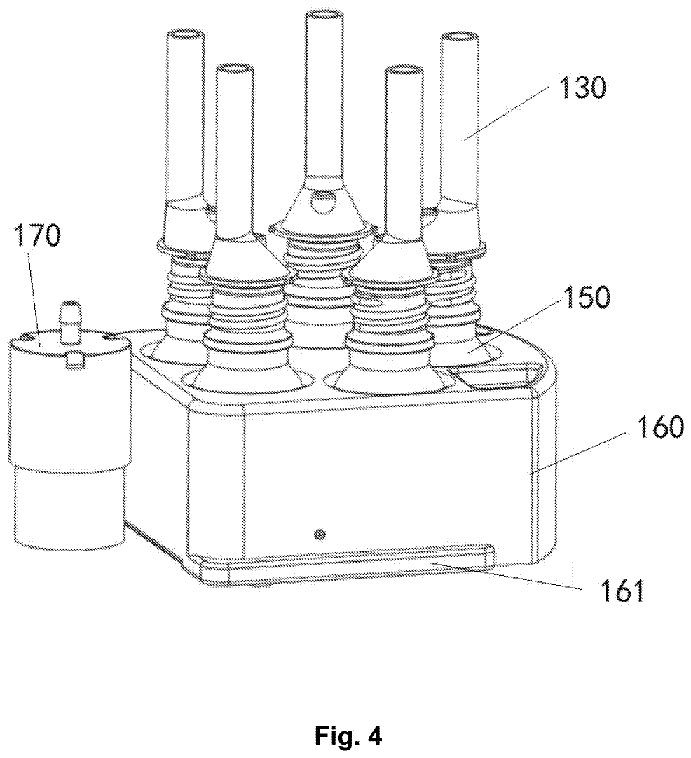

Referring to FIG. 1 and FIG. 4, in accordance with one embodiment of the present invention, a liquid diffusing system is provided by this application. The liquid diffusing system includes a diffuser for diffusing liquid. The diffuser comprises a base 10 and a platform 20. The base 10 with opening at upper end has a cavity therein for accommodating a liquid container 150, the platform 20 is configured to connect the liquid container 150 and be subject to external force from one end of the platform 20. The diffuser can be shaped differently to accommodate different design of the container 150, if desired. The container 150 is located in the cavity of the base 10 for containing liquid, including but not limited to scent, nutraceutical, pharmaceutical, flavoring, medicine. Preferably, the container 150 can be an aluminum, plastic or glass bottle.

The system further includes a chamber 130 and a nozzle 140, and the nozzle is connected with a tube 141. One end of the chamber 130 is connected to the nozzle 140 and the other end of which is connected to the platform 20. The nozzle 140 is connected with the chamber 130 for flowing air in the chamber 130 into the container 150 and sucking up liquid from the container 150. By this structure, liquid flows into the nozzle 140, and the nozzle 140 turns the liquid into micro-particle by Bernoulli's principle. Thus micro-particle of liquid is dispensed in the chamber 130 and inside container 150 that is filled with air, but liquid itself flows back to liquid part of the container 150 and the tube 141 connected to the nozzle 140.

A sealing member engaged with the chamber 130 for sealing the chamber 130 and the nozzle 140 is also provided. The sealing member has a function of sealing in the art, such as a gasket made of rubber or plastic, or a sealing ring, which aims to minimize leakage at the connection. With this sealing member, extensive manual insertion or screwing in of each liquid container can be avoided.

The system further comprises an air source, such as air actuator, connected to the platform 20 for flowing air into the chamber 130. For example, air can be pumped into the chamber 130 by an air pump having corresponding tube which is inserted into the chamber 130 with proper sealing.

After air is pumped into the chamber 130, the nozzle 140 creates low pressure by Bernoulli's principle, and the liquid flows into the nozzle 140 and turned into micro-particles by the low pressure when external force is applied on sealing between the nozzle 140 and the chamber 130, and the micro-particles are sprayed outside by holes 210 located on the platform 20. When air current is supplied, difference in pressure causes suction of the liquid from liquid container 150; and liquid that is sucked meets pressurized air in chamber 130 is dispensed the liquid into micro-particles. Further, remaining liquid in the chamber 130 is returned into the liquid container 150 via the nozzle 140.

In one embodiment of the invention, the system further comprises a locking member for locking the sealing state between the chamber 130 and the nozzle 140. And accordingly, the system further comprises a trigger like a motor for triggering the locking member to change the state between the chamber 130 and the nozzle. For example, it can change the state between the chamber 130 and the nozzle 140 from releasing to sealing, and vice versa; it can also change the state between the chamber 130 and the nozzle 140 from sealing to releasing.

In another embodiment of the present invention, the liquid diffusing system of the present invention is further provided with a tray 160 located in the cavity of the base 10 for receiving a plurality of containers 150. Of course, a plurality of chambers 130 and nozzles 140 can be provided correspondingly for cooperating with the containers 150. Preferably, the tray 160 is provided with at least one protruding member 161 at the bottom, and the base 10 is provided with at least one slot 111 for receiving the protruding members 161 correspondingly. Thus, the protruding members 161 can be inserted into the slots 111 easily. According to the present invention, the tray 160 is arranged a plurality holes 120 to accommodate a plurality of containers 150. In this embodiment, an air pump 170 is provided to supply air into all the chambers 130 in the system.

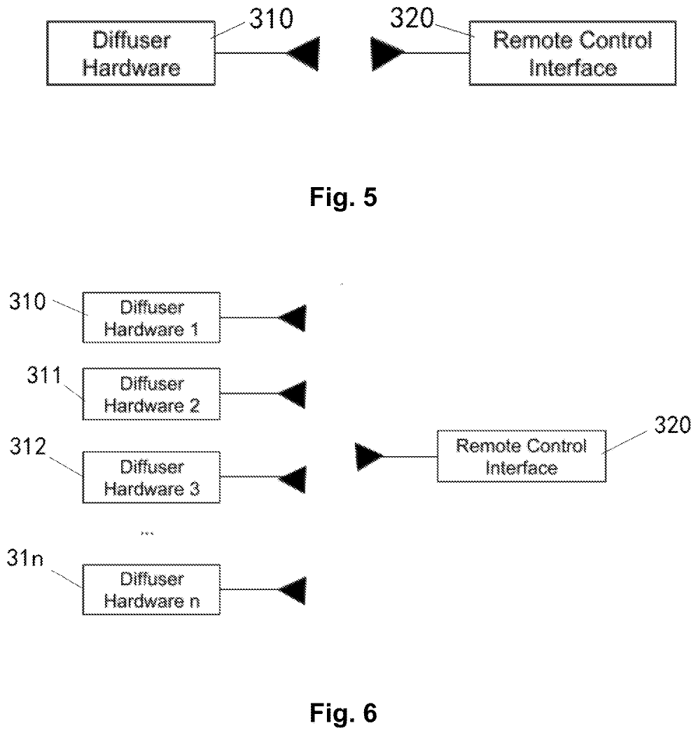

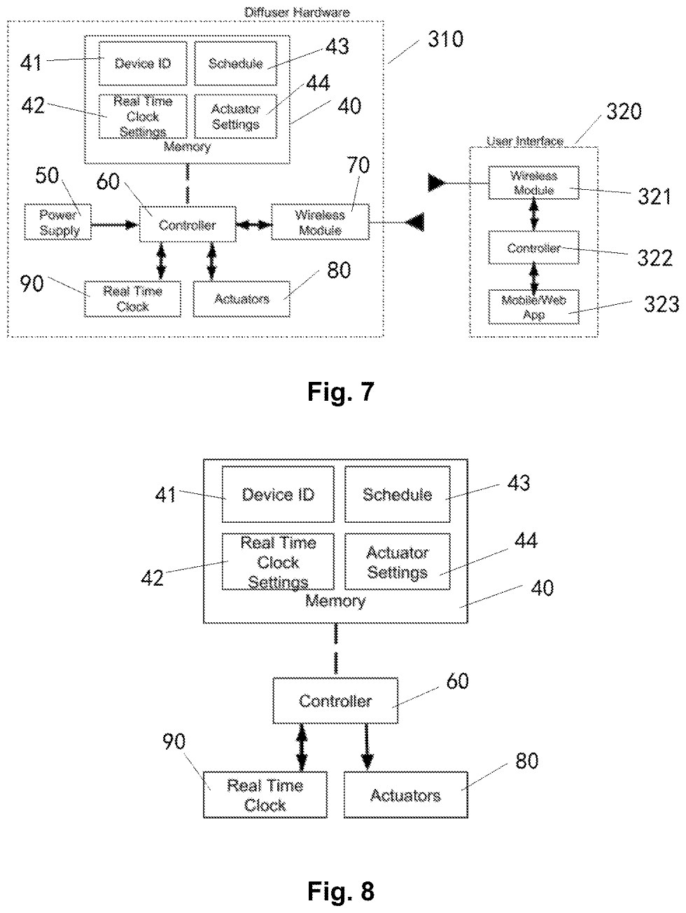

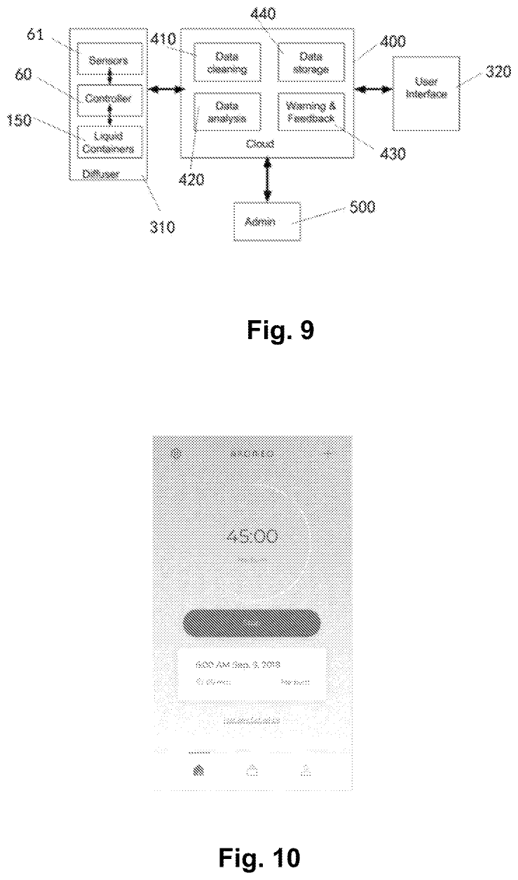

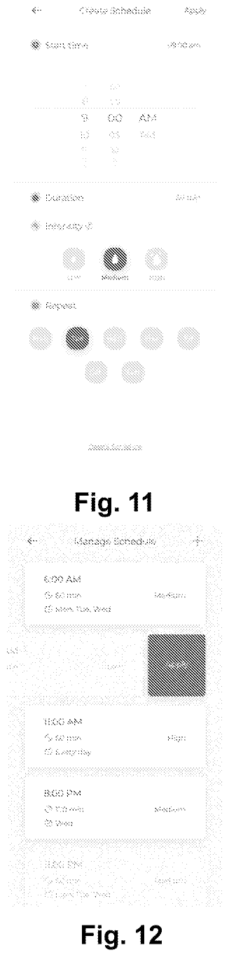

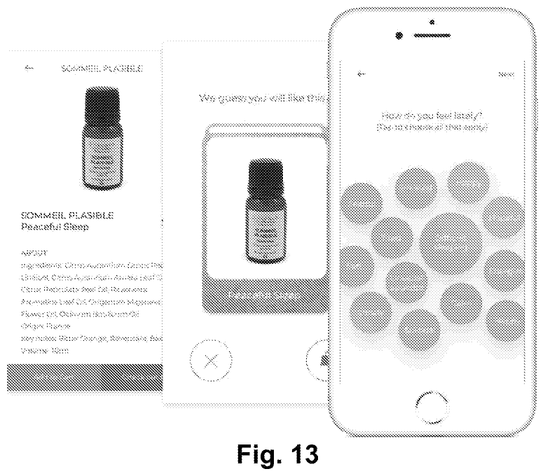

Referring to FIG. 5-FIG. 9, according to the liquid diffusing system of the present invention, the system further comprises a wireless system for controlling the liquid diffusing system easily and simply, in which the wireless system comprises a diffuser hardware 310 and a remote control interface 320, which are communicated wirelessly, such as WI-FI, Bluetooth, wireless LAN, infrared, voice, gesture and other equivalent means. If there is a need to control various diffusers 310 in the system, a plurality of diffusers 310, 311 . . . 31n can be provided, which are communicated with one remote control interface 320, as shown in FIG. 6.

The diffuser hardware 310 comprises a controller 60, a memory 40, a communication module such as wireless module 70, and an actuator 170. All the above elements can be communicated, and the controller 60 controls operation of these elements. In a preferable embodiment, the hardware 310 can also include power supply 50 for providing energy, a Real Time Clock 90 for providing time control. Particularly, the memory 40 in the system can adjust and store the following information: device ID 41, Real Time Clock Settings 42, Schedule 43 and Actuator Setting 44. All the information thereof can be stored in the memory 40 for further use.

The remote control interface 320 comprises a communication module, such as wireless module 321, a controller 322 and a controlling App such as mobile APP or Web App 323. The interface 320 is configured to communicate with the diffuser hardware 310 so as to be preprogrammed to activate and deactivate said diffuser according to a predetermined activation profile.

Preferably, in one embodiment, the wireless system is configured to control one diffuser at a time. In another embodiment, the wireless system is configured to control multiple diffusers at once.

In one embodiment of this invention, the diffuser hardware is operated by parameters which comprises frequency of liquid dispensing action to control intensity.

Further, the wireless system further comprises cloud 400 as an intermediary between the diffuser hardware 310 and the remote control interface 320. The cloud 430 can carry out functions of calculation and storage. For example, the cloud 400 can have the function of data cleaning 410, data analysis 420, warning and feedback 430 and data storage 440. Based on the above data collected by different sensors 61 in the system, the cloud 400 carry out the above functions to achieve monitoring of the system, which can track the usage of the liquid, behavior of the user when using the diffuser, condition of the environment or the status of the diffuser, for example, to monitor the system to confirm if it is working properly, or broken or clogged. The cloud then gives information of waning or positive feedback to user to avoid extra cost incurred due to problem that is have not found by user himself/herself. In fact, the cloud 400 produces feedback which could be fed to remote control interface 320 or alter Admin 500. The remote control interface 320 or an admin 500 could generate data or give command to the cloud 400, which can be transferred to the diffuser.

According to one embodiment of the invention, the sensors 61 are provided for collecting data so as to monitor the status of the diffuser or the environment. Preferably, the sensors include air quality sensor, air flow sensor, humidity sensor and load sensor. The data points of the liquid diffusing system comprise of data from sensors, timer, user input to the user interface, and user behavior when using the system.

Another embodiment of the invention is where the data collected through the diffuser hardware 310 and remote control interface 320 is feed into an algorithm, intelligent algorithm, machine learning system, or other forms of artificial intelligent systems to generate output data that is presented back to admin or user interface. The output is also stored in the cloud.

Preferably, the wireless system further comprises a light unit for providing light. More preferably, the wireless system further comprises a music unit for providing music. More preferably, the wireless system further comprises an air filter for filtering air. These units can be selectively added in the system, if desired. Those skilled in the art can have a choice to install and operate these units, which is omitted herein accordingly.

According to one embodiment of the invention, the remote control interface 320 is communicated with a timer configured to activate or deactivate the diffuser according to a user instruction. Setting of the timer is also familiar to those skilled in the art, detail of which is also omitted herein.

In light of the above, the system of the invention does not use additional material to aid in the spraying of liquid, which only adopts Bernoulli principle, a theorem of fluid dynamics that states that as the speed of air current increases, simultaneously the pressure drops. By the principle, the liquid in the container can be turned into micro-particles due to sudden decrease in pressure. Thus only providing air in the diffuser results in the operation of the diffuser.

The system can include several containers to be operated at the same time, which can generate diversity of mist in one diffuser.

The system can be operated by user through a user interface via wireless connection, which enables remote and automated control of the system and that has not been possible with diffusers in the prior art.

The invention has been described hereinabove using specific examples; however, it will be understood by those skilled in the art that various alternatives may be used and equivalents may be substituted for elements or steps described herein, without deviating from the scope of the invention. Modifications may be provided to adapt the invention to a particular situation or to particular needs without departing from the scope of the invention. It is intended that the invention not be limited to the particular implementation described herein, but that the claims be given their broadest interpretation to cover all embodiments, literal or equivalent, covered thereby. Accordingly, the above disclosure should be construed as limited only by the metes and bounds of the appended claims.

### Example 2

For the following examples, the term “cover” is used and refers to “platform” in the previous examples; the term “duct” is used and refers to “chamber” in the previous examples; the term “vent” is used and refers to “hole” as used in the previous examples; the term “recess” is used and refers to “hole”.

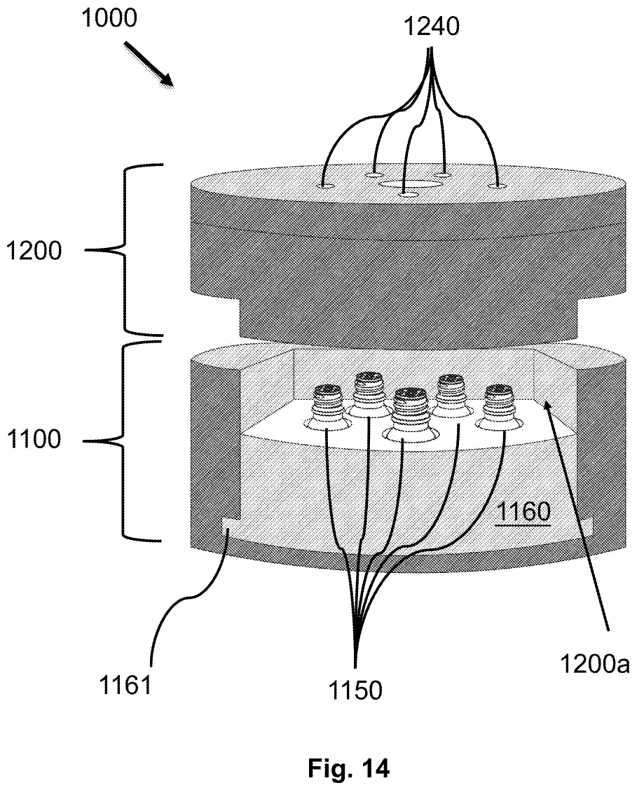

FIG. 14 is a perspective-exploded view of a liquid diffuser 1000, according to one example embodiment. The liquid diffuser 1000 includes a housing formed from a base 1100 and a cover 1200. In this implementation, the housing is substantially a cylindrical shape. In yet other example embodiments, the housing may be configured to have any possible shapes such as rectangular, cubic, hexagonal etc. and/or possible sizes.

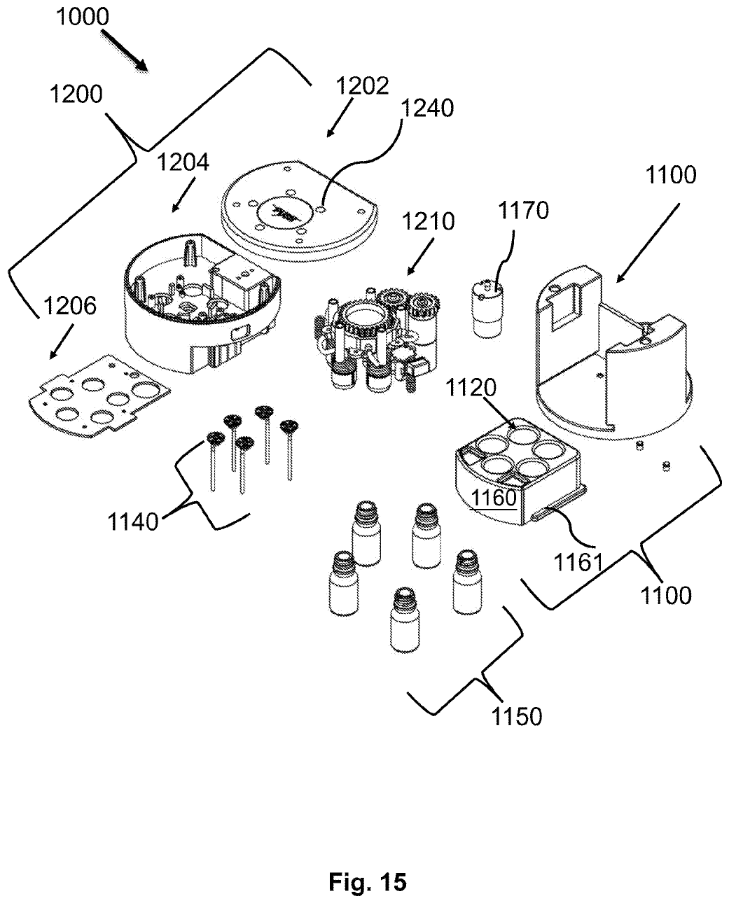

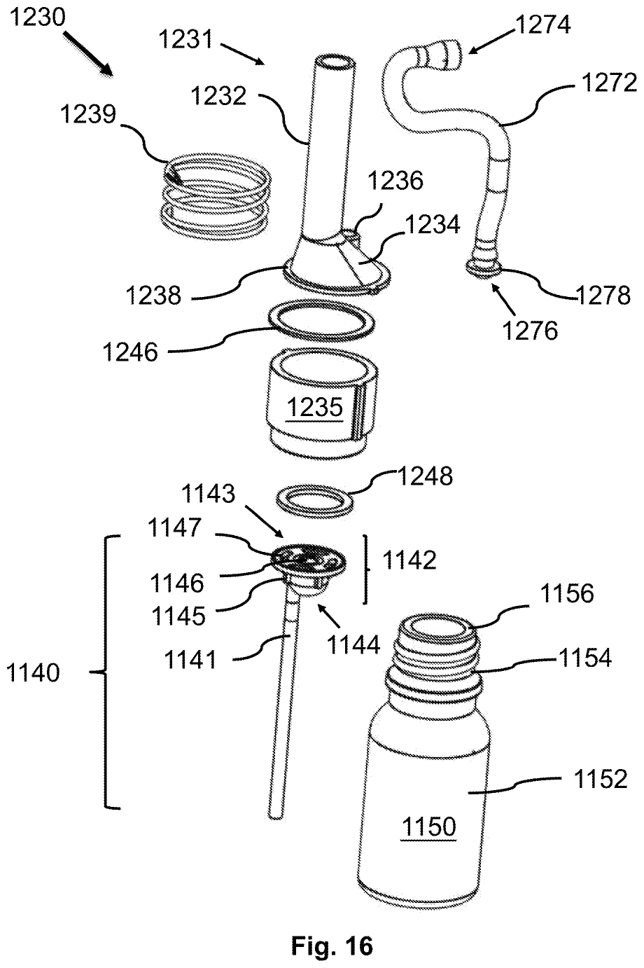

For clarity, the base 1100 is shown as being separated from the cover 1200 (as in an open state) in this figure. The base 1200 has a cavity 1200a to receive a tray 1160 that accommodates one or more liquid containers. In this implementation, the tray has five recesses sized and shaped to accommodate five liquid containers 1150 respectively such that the liquid containers 1150 are substantially secured in the recesses. Each liquid container is provided with a nozzle (only partially shown in FIG. 14 and will be shown in FIGS. 15 and 16). The tray 1160 has an outer edge forming a protruding member 1161 which is shaped to match a slot in the base 1200 such that the tray 1160 is slidably removable from the case 1200 so that a user may completely take out the tray 1160 from the base to ensure easy installation or replacement of the liquid containers. The tray 1160 is also configured to slidably mate with the base 1200 such that each liquid container within the tray 1160 is properly positioned at a designated area of the liquid diffuser 1000 to align with each of the corresponding duct. The cover 1200 has five vents 1240 extending from the side away from the base (i.e., a first side) to the opposing second side. For ease of description, the first and second sides are referred to as the top and bottom sides respectively. By way of example, the cover 1200 is sized and shaped to engage with the base 1100.

For ease of description, all subsequent figures with the same reference numerals refer to the same parts and will not be repeated in the description for each figure.

FIG. 15 a perspective-exploded view of a liquid diffuser 1000 in accordance with another example embodiment in an open state. In this implementation, the cover 1200 includes a top cover 1202, a bottom cover 1204 and a bottom plate 1206. By way of example, the top cover 1202 has five vents 1240 extends through the top cover and these vents are connected in gas communication with the corresponding ducts at their respective positions when the top cover 1202 is secured to the bottom cover 1204.

In one example embodiment, the diffuser 1000 may include one container having one nozzle and one duct. In yet other example embodiments, the diffuser may contain different numbers of containers and the corresponding duct assemblies and nozzles, such as 1, 2, 3, 4, 5, 6, 7, 8, 9, 10 or more. In one embodiment, the diffuser contains five (5) containers with five corresponding duct assemblies and nozzles.

The bottom cover 1204 is a casing having a space therein to accommodate a diffuser assembly 1210 (including, for example, ducts, trigger and gas supply line, etc., which will be described in greater detail in FIGS. 16-18), within the cover 1200. In this implementation, the liquid diffuser 1000 also has an actuator to provide a gas supply, which is a gas pump, for example, an air pump 1170. In this implementation, the air pump 1170 is disposed within the base. In yet another example embodiment, instead of housing a gas source such as an air pump in the base, a pressurized gas channel that is connectable with an external gas source may be provided. By way of example, by controlling the on-off function and the run time of the actuator, the intensity of the liquid to be diffused can be regulated. By way of example, the gas pump may be coupled to a power supply to supply energy to actuate the gas pump to supply the gas stream or to other components such as controller (not shown). In yet other example embodiments, the gas supply may be external to the diffuser 1000 and may be other gas sources such as air compressor or a motor coupled with an air compressor. Other gases such as nitrogen may be used.

In yet other example embodiments, multiple gas sources may be used and each gas supply line may have a separate gas source. For ease of description, an air pump 1170 will be used as an example for the gas source. By way of example, the containers 1150 are threaded standard essential oil glass vials for such as 5 ml, 10 ml or 15 ml liquids. In yet other example embodiments, the containers may be made of other materials such as metal or plastic and have different volumes and different closure means or designs. By way of example, the ducts may be configured to fit the specific shape or design of the container. FIG. 15 also shows five nozzles 1140 configured to fit for the five respective containers 1150. Detailed structures of the nozzles 1140 will be shown in FIG. 16.

FIG. 16 shows a more detailed perspective-exploded view of a container, a nozzle, a duct assembly and a gas supply line of a liquid diffuser 1000, in accordance to an example embodiment. In this implementation, the container 1150 has a substantially cylindrical body 1152, a reduced or narrowed neck portion 1154 having threads on the outer peripheral and an open end 1156 having a defined diameter. The nozzle 1140 has a nozzle tube 1141 connected to a nozzle head 1142 having a disc portion 1143 and a bowl portion 1144. The disc portion 1143 has a central inlet 1146 which is directly connected in gas communication with the bowl portion 1144 and the subsequent nozzle tube 1141. The disc portion 1143 also has four discontinuous slits or nozzle outlets 1147 axially surrounding the central inlet 1146. The disc portion 1143 has a diameter slightly larger than the outer peripheral of the open end 1156. The bowl portion 1144 further includes a number of ridges 1145 extending outward therefrom. The nozzle head 1142 is sized and shaped to match with space within the open end 1156 and the neck portion 1154 such that when the nozzle tube 1141 is inserted into the container 1150, the nozzle 1140 is properly secured to the container 1150 in a desired position. The nozzle 1140 may be made of plastics and/or metals.

Still referring FIG. 16, the duct assembly 1230 includes a duct 1231, and optionally a duct extension 1235 and a duct spring 1239. In this implementation, the duct 1231 includes (1) an elongated canal portion 1232 that connects in gas communication to the vent 1240 (shown in FIG. 15), (2) a cup portion 1234 that connects in gas communication between the canal portion 1232 and the extension 1235, (3) a rim 1238 that extends from the peripheral of the bottom end of the cup portion 1234 and (4) a gas inlet 1236 through which the gas stream line 1272 extends. The duct 1231 acts as the gas communication channels from the gas source to the nozzle to contact with the liquid inside the container and subsequently to the vent. By way of example, the duct 1231 may extend though the cover, that is, at least partially extend through the cover. In yet another example embodiment, the duct 1231 may not extend though the cover but instead directly or indirectly connected with the cover (and the vent). The gas inlet 1236 receives at least a portion of the gas stream line 1272 such that the gas stream from the gas source can be transferred through the gas stream line 1272 to the nozzle inlet 1146. The extension 1235 is substantially cylindrical and has a top open end having a diameter substantially the same as the rim 1238 and a reduced or narrowed bottom open portion having a diameter substantially the same as the nozzle disc portion 1143. The extension 1235 acts as an adaptor between the duct 1231 and the nozzle 1140. Optionally, the extension 1235 may be provided with a sealing member 1246 to seal the contacting surfaces between the rim 1238 and the top open end of the extension 1235, and/or a sealing member 1248 to seal the contacting surfaces between the reduced or narrowed portion of the extension 1235 and the peripheral of nozzle disc portion 1143 to avoid potential gas leakage. The top sealing member 1246 may have a size and shape that matches with the rim 1238 and the top open end of extension 1235, while the bottom sealing member 1248 may have a size and shape that matches with the bottom open end of the extension 1235 and the peripheral of the nozzle disc 1143. The gas stream line 1272 has an inlet 1274 from the gas source and an outlet 1276 to the nozzle. The outlet 1276 may be optionally provided with a gas stream line sealing member 1278 to avoid potential gas leakage. By way of example, the sealing members may be in the form of a gasket or a ring and may be made of rubber or plastics. The gas stream line 1272 connects in gas communication between a gas source and the nozzle inlet 1146. The duct assembly 1230 may additionally include a duct spring 1239 having a transverse cross-sectional diameter slightly smaller than the outer peripheral diameter of the rim 1238 so that the duct spring 1239 surrounds substantially the cup portion bottom 1234 of the duct 1231. The spring 1239 is positioned within the bottom cover (not shown in FIG. 16. See FIG. 15) and ensures a good contact between the duct assembly 1230 (or the duct extension 1235) and the nozzle 1140 when the cover 1200 and the base 1100 is positioned in the sealed state.

In yet another example embodiment, the duct may be directly connected with the nozzle without the duct extension (or duct spring), as shown in FIG. 4. In this implementation, a sealing member may be provided to the duct to seal the contacting surfaces between the duct rim and the nozzle.

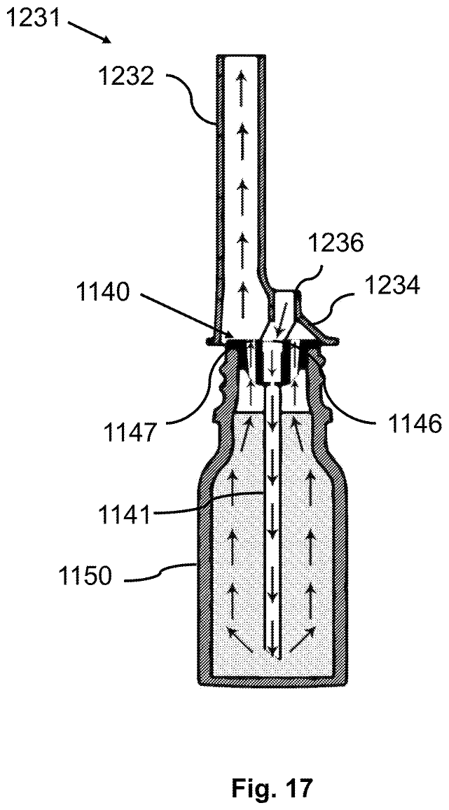

Referring now to FIG. 17, which is a cross-sectional side view of a nozzle 1140 and a duct 1231 in another example embodiment, the figure shows how the gas stream flows from the gas stream line (not shown) into and out from the duct 1231, indicated by the arrows as shown. In this implementation, the duct 1231 is connectable in gas communication with the nozzle 1140 during a sealed state when the cover 1200 is engaged with the base 1100 forming a sealed state. In other words, the duct 1231 is directly engaged in gas communication with the nozzle 1140 when the cover 1200 and the base 1100 is positioned in a sealed state. The gas stream passes through the gas stream line 1272, entering the duct gas inlet 1236 and entering the nozzle inlet 1146. Then, the gas stream flows through the tube 1141 and reaches the bottom portion of the container 1150. The gas stream is in direct contact with the liquid inside the container 1150 and exerts a positive pressure on the liquid. Without being bound by a theory, fine liquid particles or micro-particles (or mist) will be generated via the nozzle outlets 1147 and will be carried by the gas stream from the nozzle outlets or slits 1147 to the duct canal 1232 of the duct 1232 and eventually to the vent 1240 (not shown). Excessive liquid disposed in the duct canal may return to the container 1150 via the nozzle when the diffuser is not in operation (i.e., the gas source is not powered or disconnected).

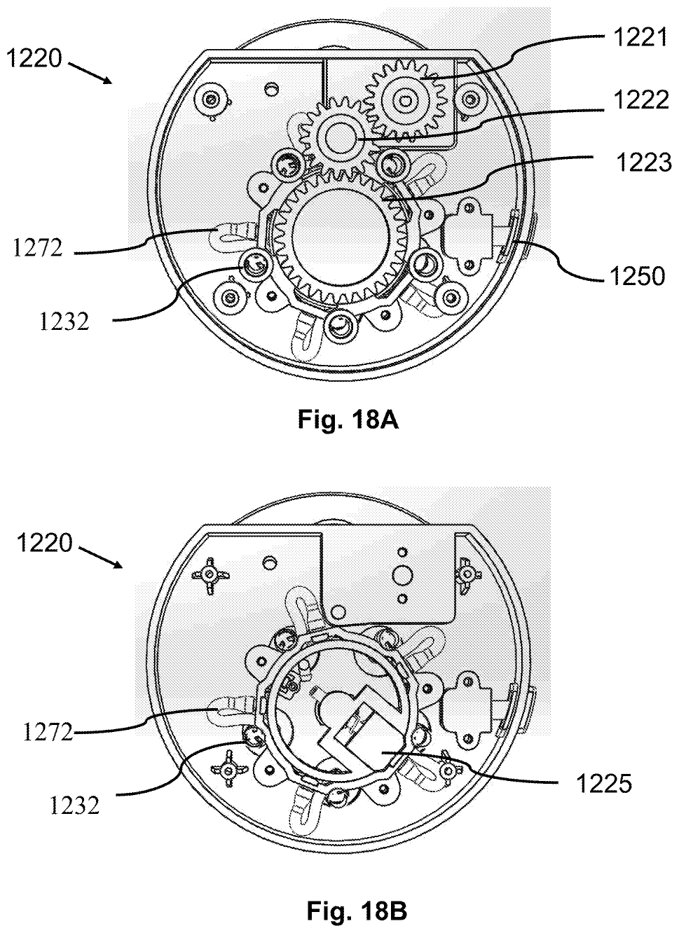

Referring now to FIGS. 18A and 18B, which are different cross-sectional transverse views of a liquid diffuser in accordance with one example embodiment, the figure shows the detailed structures of a trigger which controls and directs a gas stream to at least one designated nozzle. In this example, the trigger is a switch assembly 1220. In the implementation, provided is one single gas source (not shown) shared by five liquid containers (not shown) and the corresponding five duct assemblies (partially shown as duct canals 1232). Each gas steam line is sourced from a single gas pump. The switch assembly 1220 includes a motor (not shown), a first gear 1221, a second gear 1222 and a third gear 1223, as shown in FIG. 18A. The first gear 1221, the second gear 1222 and the third gear 1223 are substantially aligned in the same transverse plane. The third gear 1223 has more teeth than the other gears. The first gear 1221 may be actuated by the motor (not shown). The motor may rotate the first gear 1221 in a first direction which engages the teeth of the second gear 1222 and rotates the second gear 1222 in an opposing second direction and in turn the third gear 1223 receives rotary motion from the second gear 1222 in the first direction. Therefore, the rotational actuation of the motor is transmitted to the third gear 1223 with an amplified input torque. The third gear further includes an L-shaped conduit (not shown) which receives a continuous gas stream from the gas source (not shown) and directs the gas stream to the spring-loaded switch 1225, as shown in FIG. 18B. The rotation of the third gear 1223 drives the rotation of the spring-loaded switch 1225 such that the gas source may be in gas communication with any one of the five gas stream line inlets 1274, for example, in response to an input from a user interface such as a specific physical button being pressed by a user or a predetermined setting or schedule from a remote user interface. In such configuration, only one gas stream line is connectable in gas communication with the gas source at the same time, that is, only one liquid from one container is diffused at the same time. If diffusion of more liquids are desired, the spring-loaded switch 1225 may keep switching its positions to connect between two or more desired gas stream lines. By controlling the actuator for regulating the gas stream, and/or the trigger for selecting the designated nozzle, the intensity of the liquid diffusion and choice of the liquids to be diffused can be controlled. The switch assembly 1220 may include an anti-reverse mechanism to allow rotation of the drive gears in only one direction. In some embodiments, the diffuser 1000 may also include a gas flow sensor (not shown). In such embodiments, the gas stream from the gas source passes through the gas flow sensor before passing through the L-shaped conduit (not shown) and subsequent spring-loaded switch 1225. The control of the actuator may be further regulated in response to the signals from the gas flow sensor.

In yet another example embodiments, multiple gas sources may be provided and each gas stream line is supplied with a separate gas source/gas pump. In such implementation, controlling the gas sources/the actuation of gas pumps can control the intensity of liquid diffusion and choice of the liquid to be diffused. A trigger or a switch assembly then may be unnecessary.

The diffuser 1000 may further comprising one or more locking members (not shown) to connect between the cover and the base. In some embodiments, the cover is engageable the base via the one or more reversible locking members disposed on the cover and/or the base. The locking members are configured to bias the cover between an open state in which the one of more liquid containers are accessible and a sealed state in which the base and the cover are in gas communication. In certain embodiments, the locking members bias the cover (and the ducks) away from the base (and the nozzles) in a defined distance in the open state. The one of more liquid containers are freely accessible by the user so that changing the liquid containers become easy. When a user exerts a force pressing against the cover towards the base, the locking members bias the cover to engage with the base to become a sealed state such that the ducts in the cover are connectable in gas communication with the nozzles and the gas stream line in the base. A button 1250 (as shown in FIG. 18A) may be provided to the diffuser 1000 to release the locking member from the sealed state to the open state.

In some example embodiments, the diffuser 1000 may also couple to a power supply for providing energy to, for example, the gas pump or the controller or the like.

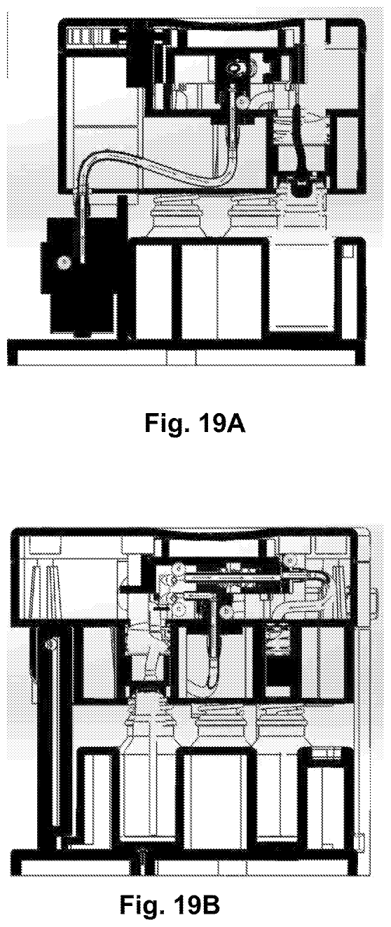

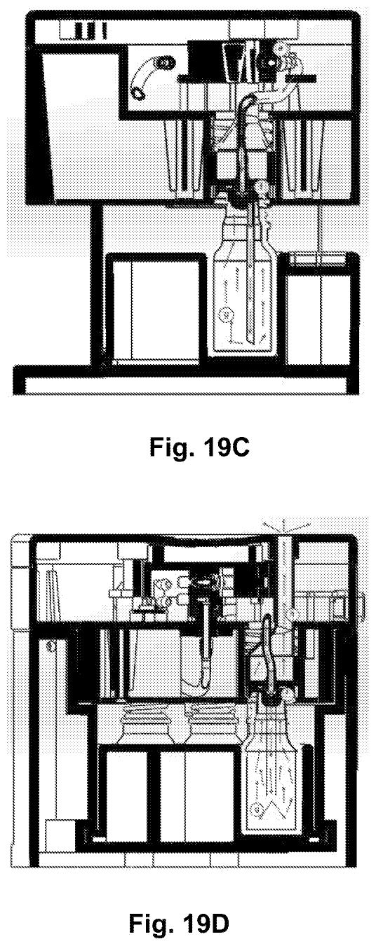

Now turning to the operation of the diffusers described above, FIG. 19A-19E shows how the diffuser 1000, according to an example embodiment, may work and how the gas stream flows during operation. For ease of description, the flow of the gas stream is indicated by arrows and certain specific locations where the gas stream flows through are represented in circled alphabets (i.e., from a to h). FIG. 19A is a cross sectional side view showing how the gas stream enters from the air pump (a) the central gas line (b). During operation (when the diffuser is in a sealed state), the gas source (which is an air pump disposed in the base in this implementation) is actuated to generate a gas stream (for example, an pressured air flow) flowing from the air pump to the central gas line in response to either a manual inputted instruction or a predetermined setting or schedule from a user interface. FIG. 19B is another cross-sectional side view showing how the gas stream from the central gas line (b) enters the gas flow sensor from the gas flow sensor inlet (c) and subsequently leaves the gas flow sensor outlet (d) and enters the switch assembly. In this implementation, the gas flow sensor is an air flow sensor that can sense the velocity of the air stream flow. Then the switch assembly directs the gas stream to the spring-loaded switch (e) to the desired gas stream line (and thus the desired liquid container). FIG. 19C shows how the gas stream leaves from the spring-loaded switch (e) via the nozzle (f) to the liquid container (g). The gas from the gas stream line flows into the liquid bottle through nozzle inlet and enters the nozzle tube. The gas then is in direct contact with the liquid inside the liquid container (g). FIG. 19D shows how the gas stream carrying the liquid micro-particles exits from the nozzle outlet to the duct (h). The liquid micro-particles together with the gas form a mist and leaves the duct canal and the vent to the surroundings.

### Example 3

For the following examples, the terms “electronic device” or “mobile electronic device” are used and refers to “remote control interface” in the previous examples; the term “communication module” is used and refers to “transceiver”; the term “wireless transmission module” is used and refers to “wireless module”.

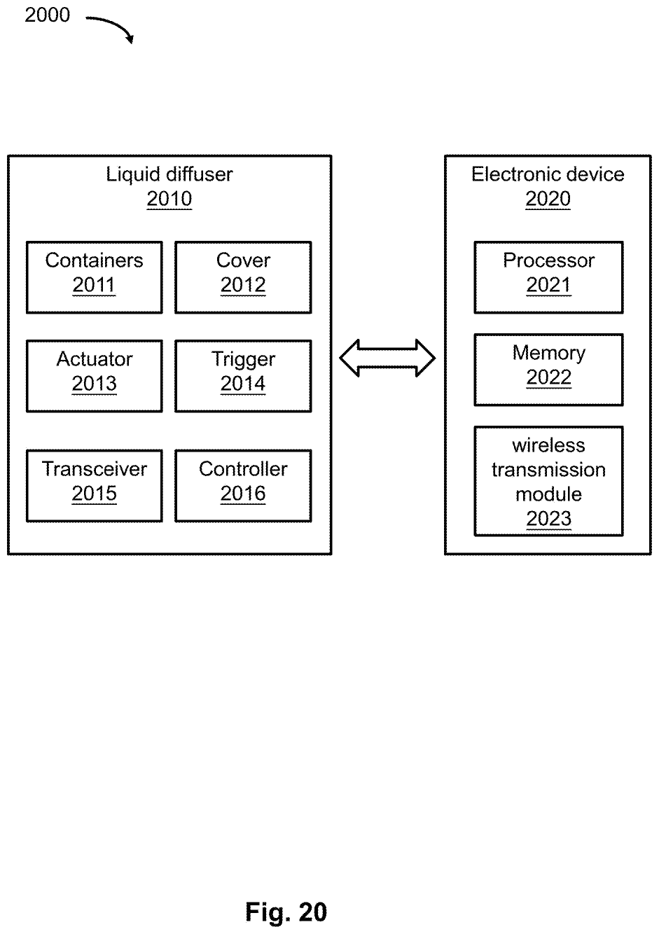

FIG. 20 shows a liquid diffusing system 2000 according to an example embodiment. As shown in FIG. 20, the liquid diffusing system 2000 includes a liquid diffuser 2010 and an electronic device 2020. The electronic device 2020 includes a processor 2021, a memory 2022 and a wireless transmission module 2023. The liquid diffuser 2100 communicates with the electronic device 2020 wirelessly.

By way of example, the electronic device 2020 may be mobile phones, portable computers, electronic pads, tablets, smart watches, remote controls, or any other electronic device that can perform wireless communication.

The liquid diffuser 2100 includes one or more containers 2011, a cover 2012, an actuator 2013, and a trigger 2014. The containers 2011 are for containing liquid such as aroma oil. Each container 2012 has one nozzle for gas flow. The cover 2012 includes one more vents that corresponds to the nozzles, for example, each vent corresponds to one nozzle. The actuator 2013 generates a gas stream, and the trigger 2014 controls and directs the gas stream to at least one designated nozzle, so that the aroma in the designated container(s) with the designated nozzle can be released to the surrounding environment. The liquid diffuser 2100 further includes a transceiver 2015 for receiving wireless signals and a controller 2016 for controlling the actuator 2013 and the trigger 2014.

When the liquid diffusing system 2000 is working, the user can send a command via the electronic device 2020 to the liquid diffuser 2010 by wireless transmission. By way of example, the command is received via a user interface in the electronic device 2020 and stored in the memory 2022. The processor 2021 processes the command into a command signal and sends the command signal by the wireless transmission module 2023 to the liquid diffuser 2010. The transceiver 2015 in the liquid diffuser 2010 receives the command signal and send the command signal to the controller 2016. Based on the command signal, the controller 2016 generates control signals to control the actuator 2013 and the trigger 2014.

By way of example, the actuator 2013 is controlled by the control signal to change its ON/OFF time period or frequency. The gas stream that the actuator 2013 generates changes accordingly, resulting in a change of the intensity of the liquid diffusion. By way of example, the trigger 2014 is controlled by the control signal to switch the designating nozzle, so that the gas stream can be directed to one designated nozzle or another.

Each nozzle belongs to one container and corresponds to one vent in the cover, and the gas stream can be directed to different containers according to the control signal. Therefore, if each container contains a different type of liquid, such as different aroma oils with different kinds of fragrances, then the fragrance to be diffused into the environment via the vent can be selected and controlled by the electronic device 2020 wirelessly.

In one example embodiment, the electronic device 2020 stores a group of pre-determined settings for the actuator 2013 and the trigger 2014, so that the user can choose a pre-determined setting and send it to the liquid diffuser 2010.

In one example embodiment, the pre-determined settings are further associated with a time schedule, so that the actuator 2013 and the trigger 2014 operate according to the time. For example, according to one pre-determined setting, the trigger 2014 is directed to a first designated nozzle and the actuator is set to a first ON/OFF frequency from 7:00 am to 5:00 pm, so that a first aroma is released into the surrounding environment at a first intensity; the trigger 2014 is directed to a second designated nozzle and the actuator is set to a second ON/OFF frequency from 5:00 pm to 10:00 pm; the trigger 2014 is directed to a third designated nozzle and the actuator is set to a third ON/OFF frequency from 10:00 pm to 7:00 am, so that a third aroma is released into the surrounding environment at a third intensity. By way of example, the time is provided by a clock in the controller 2016.

In one example embodiment, the trigger 2014 is controlled to switch between one or more nozzles, so that a combination of fluids from more than one containers 2101 is diffused in the environmental air.

In one example embodiment, the liquid diffuser 2010 and the electronic device 2020 are connected by a wireless network, which includes but is not limited to WIFI, Bluetooth, cellular network and other kinds of wireless networks.

In one example embodiment, the liquid diffuser 2010 further comprises a real time clock module (not shown) for providing time control.

In one example embodiment, the liquid diffuser 2010 further comprises a memory (not shown). By way of example, the memory may store one or more of the following information: device identity, real time clock module settings, predetermined schedule and actuator settings.

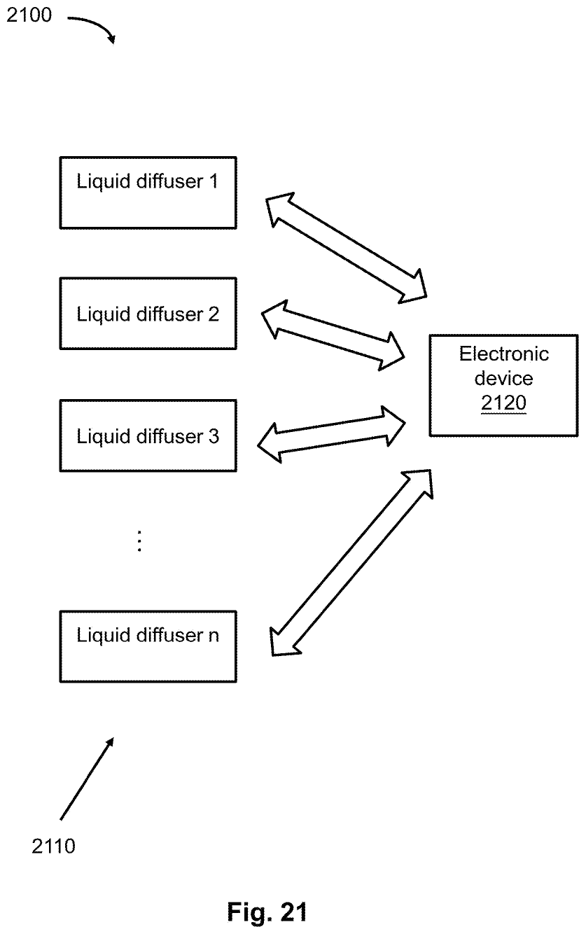

FIG. 21 shows a liquid diffusing system 2100 according to an example embodiment.

The liquid diffusing system 2100 includes n (n is an integer that is larger than 1) liquid diffusers 2110. Each liquid diffuser has a unique identity and can communicate with the electronic device 2120 wirelessly. By way of example, the electronic device 2120 and each liquid diffuser have structures that are described in FIG. 20.

In one example embodiment, the electronic device 2120 identifies the liquid diffusers 2110 by its identity and send control signals to each liquid diffuser respectively, so that the electronic device 2120 can control multiple liquid diffusers 2110 that are located at different positions with different settings.

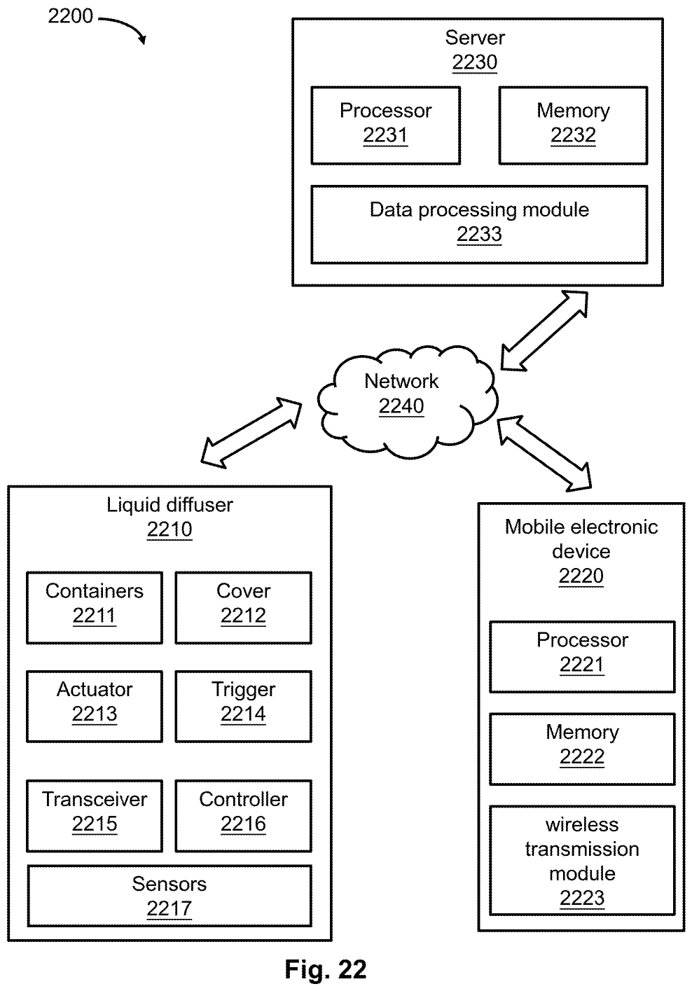

FIG. 22 shows a liquid diffusing system 2200 according to an example embodiment.

The liquid diffusing system 2200 includes a liquid diffuser 2210, a mobile electronic device 2220 and a server 2230 which communicate with each other via a network 2240. The liquid diffuser 2210 includes containers 2211, a cover 2212, an actuator 2213, a trigger 2214, a transceiver 2215, a controller 2216, which is similar to the liquid diffuser 2010 in FIG. 20. The liquid diffuser 2210 further includes a plurality of sensors 2217 for acquiring parameters of the liquid diffuser 2210 and its surrounding environment.

The mobile electronic device 2220 includes a processor 2221, a memory 2222 and a wireless transmission module 2223. The server includes a processor 2231, a memory 2232 and a data processing module 2233.

In one example embodiment, the mobile electronic device 2220 includes an interface (not shown). By way of example, the interface is a mobile App for manual input the parameters or settings.

In one example embodiment, the liquid diffuser 2210 acquires information by the sensors 2217 and send the information to the server 2230 for processing. The server 2230 stores the information in the memory 2232 and the processor 2231 performs algorithms to analyze the data. The server 2230 can send message to the mobile electronic device 2220 when necessary, for example, it determines that the liquid diffuser 2210 is not working properly.

In one example embodiment, the sensors 2217 includes but are not limited to air flow sensors for measuring the flow speed of the gas stream line of the liquid diffuser 2210, sensors for measuring the humidity, temperature and illumination of the environment, sensors for monitoring the fluid level in the containers 2211, sensors for evaluating the air quality of the environment, sensors for measuring the load or weight of the containers and sensors for tacking the behavior of the users.

In one example embodiment, the server 2230 analyzes the data obtained from the sensors and transmits the analyzing result to the mobile electronic device 2220, so that the user can refer to the information and change the settings of the liquid diffuser 2210 accordingly. By way of example, if air flow sensors detects a reduced or low flow speed, the server 2230 analyses the data and transmits the warning message to the mobile electronic device 2220.

In one example embodiment, the liquid diffusing system 2200 includes more than one liquid diffuser 2210, so that the data for more than one liquid diffuser can be collected and analyzed by the server 2230. For example, each liquid diffuser is for one user, so the using habit of each user can be analyzed from the data collected. For example, a plurality of liquid diffusers distributed in different locations belongs to one user, so the using habit of this user can be analyzed from the data collected.

In one example embodiment, the liquid diffusing system 2200 further include a light unit that electrically communicates with the server 2230. The light unit is controlled to emit light in response to a command received from the server, for example, based on a pre-determined schedule or the analysis of the data processing module 2233.

In one example embodiment, the liquid diffusing system 2200 further include a music unit that electrically communicates with the server 2230. The music unit is controlled to play music in response to a command received from the server, for example, based on a pre-determined schedule or the analysis of the data processing module 2233.

In one example embodiment, the liquid diffusing system 2200 further include an air filter that electrically communicates with the server 2230. The air filer is controlled to turn on in response to a command received from the server, for example, based on a pre-determined schedule or the analysis of the data processing module 2233.

In one example embodiment, the light unit, the music unit and the air filter are embedded in the liquid diffuser 2210.

In one example embodiment, the light unit, the music unit and the air filter are controlled based on command from the mobile electronic device 2220.

In one example embodiment, the server 2230 is a cloud server.

Blocks and/or methods discussed herein can be executed by a software application, an electronic device, a computer, firmware, hardware, a processor, or a computer system. Furthermore, blocks and/or methods discussed herein can be executed automatically with or without instruction from a user.

The method and apparatus in accordance with example embodiments are provided as examples, and examples from one method or apparatus should not be construed to limit examples from another method or apparatus. Further, methods and apparatus discussed within different figures can be added to or exchanged with methods and apparatus in other figures. Further yet, specific numerical data values (such as specific quantities, numbers, categories, etc.) or other specific information should be interpreted as illustrative for discussing example embodiments.

What is claimed is:

1. A liquid diffuser, comprising
a base having a cavity therein to accommodate at least one liquid container with at least one nozzle; and
a cover;
at least one duct extending through the cover;
each duct being connectable in gas communication to each nozzle;
wherein each duct further comprises a gas stream line connectable from a gas source to the corresponding nozzle such that a gas stream is in direct contact with a liquid inside the liquid container to create a mist via the nozzle.

2. The liquid diffuser of claim 1, wherein the cover is engageable with the base via one or more locking members configured to bias the cover between an open state in which the at least one liquid container is accessible and a sealed state in which the base and the cover are in gas communication.

3. The liquid diffuser of claim 1, wherein each gas stream line is connected in gas communication to a separate actuator.

4. The liquid diffuser of claim 1, further comprising a single actuator connectable to the gas stream lines.

5. The liquid diffuser of claim 4, further comprising a trigger to selectively control and direct the gas stream from the actuator to at least one designated nozzle.

6. The liquid diffuser of claim 5, further comprising a controller to control the actuator for regulating the gas stream, and/or the trigger for selecting the designated nozzle.

7. The liquid diffuser of claim 1, wherein each duct further comprises a sealing member to seal at least a portion of the nozzle.

8. The liquid diffuser of claim 1, wherein the base further comprises a slidably removable tray to receive the at least one liquid container.

9. The liquid diffuser of claim 1, wherein
the cover further comprises at least one vent;
each duct connects in gas communication with a corresponding vent such that the mist is diffused through the corresponding vent.

10. A liquid diffuser, comprising
a base having a cavity therein to accommodate at least one liquid container with at least one nozzle;
a cover that includes one or more ducts that corresponds to the nozzles;
an actuator for generating a gas stream;
a trigger for controlling and directing the gas stream to at least one designated nozzle;
a wireless transceiver for receiving the command signals from an electronic device via a network; and
a controller for generating control signals based on command signals received from an electronic device,
wherein the control signals are provided to the actuator for regulating the gas stream, and/or to the trigger for selecting the designated nozzle, so that intensity of liquid diffusion and choice of the liquid to be diffused can be controlled remotely.

11. The liquid diffuser of claim 10, further comprises one or more sensors for sensing one or more parameters of the environment and/or status of the liquid diffuser; wherein the controller generates the command signals based on the parameters.

12. The liquid diffuser of claim 11, wherein the sensors include one or more of an air quality sensor, an air flow sensor, a humidity sensor, a temperature sensor and a load sensor.

13. A liquid diffusing system, comprising:
an electronic device for sending command signals via a network; and
at least one liquid diffuser, wherein each liquid diffuser has an unique identity and comprises:
one or more liquid containers, each container having one nozzle;
a cover comprising one or more vents that correspond to the nozzles;
an actuator for generating a gas stream;
a trigger for controlling and directing the gas stream to at least one designated nozzle;
a wireless transceiver for receiving the command signals from the electronic device via the network; and
a controller for generating control signals based on the command signals,
wherein the control signals are provided
to the actuator for regulating the gas stream, and/or
to the trigger for selecting the designated nozzle,
so that intensity of liquid diffusion and choice of the liquid to be diffused can be controlled remotely.

14. The liquid diffusing system of claim 13, wherein the liquid diffuser further comprises one or more sensors for sensing one or more parameters of the environment and/or status of the liquid diffuser; wherein the controller generates the command signals based on the parameters.

15. The liquid diffusing system of claim 14, wherein the sensors include one or more of an air quality sensor, an air flow sensor, a humidity sensor, a temperature sensor and a load sensor.

16. The liquid diffusing system of claim 13, further comprising:
a server,
wherein the server communicates with the electronic device and with the liquid diffuser;
and wherein the server
receives data from the electronic device and/or the liquid diffuser, analyzing the data; and
provides feedback to the electronic device and/or the liquid diffuser.

17. The liquid diffusing system of claim 13, wherein the electronic device further comprises:
a processor;
a memory that stores one or more groups of pre-determined command signals for the liquid diffuser; and
a wireless transmission module;
wherein the processor determines one or more groups of pre-determined command signals based on a time schedule and the wireless transmission module sends the one or more groups of pre-determined command signals to at least one liquid diffuser.

18. The liquid diffusing system of claim 13, further comprising
one or more additional devices that communicate with the electronic device,
wherein the additional device is a light unit, a music unit and/or an air filter;
wherein the one or more additional devices are activated in response to the command signals received from the electronic device.
